(19) United States
(12) Patent Application Publication
(10) Pub. No.: US 2008/0031932 A1
(43) Pub. Date: Feb. 7, 2008

(54) Transdermal atomoxetine formulations and associated methods

(75) Inventor: Kamal K. Midha, London (GB)

Correspondence Address: THORPE NORTH & WESTERN, P.O. BOX 1219, SANDY, UT, 84091-1219, US

(73) Assignee: Watson Laboratories, Inc., Salt Lake City, UT (US)

(21) Appl. No.: 11/499,912

(22) Filed: Aug. 4, 2006

(51) Int. Cl. A61K 31/138; A61K 9/70

(52) U.S. Cl. 424/449; 514/651

(57) ABSTRACT

Methods and formulations for delivering atomoxetine compounds that minimize drug metabolism and thus increase the effectiveness of the drug are disclosed. The method may include maximizing the in vivo potency of an atomoxetine compound in a subject by transdermally administering the atomoxetine compound to the subject. The in vivo potency of the atomoxetine compound may be maximized by minimizing the in vivo conversion of the atomoxetine compound to an atomoxetine compound metabolite.

### FIELD OF THE INVENTION

The present invention relates to transdermal atomoxetine formulations and methods that increase in vivo atomoxetine potency in a subject. Accordingly, this invention involves the fields of chemistry, pharmaceutical sciences, medicine and other health sciences.

### BACKGROUND OF THE INVENTION

Drug metabolism can often be problematic to the administration of various pharmaceuticals, both in terms of decreasing the amount of active drug available to exert a therapeutic effect, and due to metabolic variability between individuals. These variable pharmacological effects between individuals can create dosing challenges, particularly for those drugs that affect behavior or those that require fairly specific blood serum level ranges.

One potential solution to compensate for lower pharmacological effects is to increase the administered dosage of the drug. In addition to increasing serum levels of the active drug, however, increasing the administered dosage also tends to increase the concentration of drug metabolites in the blood due to increased drug metabolism for drugs which follow linear pharmacokinetics. As such, this approach may result in increased side effects for some drugs.

Metabolic variability between individuals can also be problematic to the administration of many drugs, particularly for those that need to be delivered to have a precise range of blood serum levels. In these cases, blood serum levels between individuals that metabolize the drug at different rates can vary dramatically. Those that metabolize quickly will experience a rapid decline in blood serum levels, while those that metabolize more slowly retain higher levels of the drug for much longer periods. As such, it can be difficult to prescribe and monitor the therapeutic actions of a drug across individuals.

Atomoxetine is one drug that may exhibit such metabolic problems associated with its administration. Atomoxetine is a selective norepinephrine reuptake inhibitor (SNRI) that is often used in the treatment of attention-deficit/hyperactivity disorder (ADHD), and is commercially available as the oral formulation Strattera® from Eli Lilly Co. The precise mechanism by which atomoxetine exerts its effects in ADHD is unknown, however ex vivo uptake and neurotransmitter depletion studies suggest that it may be related to selective inhibition of the pre-synaptic norepinephrine transporter.

Atomoxetine is metabolized primarily by oxidative metabolism through the cytochrome P450 2D6 (CYP2D6) enzymatic pathway and subsequently eliminated through glucuronidation. At least two phenotypes of drug metabolism associated with CYP2D6 have been identified in the population, one exhibiting normal activity and one exhibiting reduced activity. In individuals having normal activity in the CYP2D6 pathway, atomoxetine has a plasma half-life of about 5 hours. In individuals that are part of the fraction of the population that have reduced activity in the CYP2D6 pathway, and thus are poor metabolizers of the drug, atomoxetine has a half-life of about 24 hours. As such, the administration of atomoxetine can be difficult without prior testing of individuals to determine the rate at which they metabolize through the CYP2D6 enzymatic pathway.

In view of the foregoing, compositions and methods for administering atomoxetine that reduce problems associated with drug metabolism are continuously being sought and are extremely desirable.

### SUMMARY OF THE INVENTION

Accordingly, the present invention provides formulations and methods for delivering atomoxetine compounds that minimize drug metabolism and thus increase the effectiveness of the drug. In one aspect, such a method may include maximizing the in vivo potency of an atomoxetine compound in a subject by transdermally administering the atomoxetine compound to the subject. Though various mechanisms are possible, in one aspect the in vivo potency of the atomoxetine compound may be maximized by minimizing the in vivo conversion of the atomoxetine compound to atomoxetine compound metabolites. Such atomoxetine compound metabolites may include, without limitation, 4-hydroxyatomoxetine, 4-hydroxyatomoxetine-O-glucuronide, N-desmethylatomoxetine, and combinations thereof.

A variety of atomoxetine compounds are contemplated for use in aspects of the present invention, including, without limitation, atomoxetine, 4-hydroxyatomoxetine, N-desmethylatomoxetine, and their metabolites, derivatives, salts, prodrugs, analogs, and combinations thereof. In one aspect, the atomoxetine compound may be any atomoxetine compound blocked at the phenoxy 4 position.

The potency of an atomoxetine compound may also be enhanced by administering a P450-mediated reaction inhibitor to the subject. Though various P450-mediated biotransformation inhibitors may prove to be useful in increasing potency when administered in association with an atomoxetine compound, an inhibitor of the CYP2D6 enzymatic pathway may be particularly effective. A P450-mediated reaction inhibitor can be administered either prior to, concurrently with, or following the atomoxetine compound. Such an inhibitor may also be administered both prior to and following the atomoxetine compound. Also, in one aspect the P450-mediated biotransformation inhibitor and the atomoxetine compound may be administered as a single composition.

In another aspect of the present invention, a transdermal atomoxetine formulation for use as recited herein is also provided. Such a formulation may include a therapeutically effective amount of an atomoxetine compound in combination with a pharmaceutically acceptable transdermal carrier, wherein the administration of the combination to a subject may maximize the in vivo potency of the atomoxetine compound by minimizing metabolism thereof.

The amount of the atomoxetine compound included in the formulation may vary depending on a number of criteria, such as the particular atomoxetine compound to be delivered, the chemical makeup of the carrier, etc. In one aspect, however, the atomoxetine compound may be from about 0.1% w/w to about 50% w/w of the transdermal formulation. In another aspect, the atomoxetine compound may be from about 1% w/w to about 20% w/w of the transdermal formulation. In yet another aspect, the atomoxetine compound may be about 5% w/w of the transdermal formulation.

The pharmaceutically acceptable carriers of the present invention may be any carrier known to one skilled in the art. In one aspect, the pharmaceutically acceptable carrier may be a biocompatible polymer. Biocompatible polymers may include, without limitation, rubbers; silicone polymers and copolymers; acrylic polymers and copolymers; and mixtures thereof. In one aspect, the biocompatible polymer can be a rubber, which may be any useful rubber known to one skilled in the art, including natural and synthetic rubbers; plasticized styrene-rubber block copolymers, etc., and mixtures thereof. In another aspect, the biocompatible polymer may include silicone polymers, polysiloxanes, and mixtures thereof. In yet another embodiment, the biocompatible polymer may include acrylic polymers, polyacrylates, and mixtures thereof. In a further embodiment, the biocompatible polymer may include vinyl acetates, ethylene-vinyl acetate copolymers, polyurethanes, plasticized polyether block amide copolymers, and mixtures thereof.

The present invention also contemplates liquid reservoir system (LRS) formulations. Thus, in one aspect, the pharmaceutically acceptable carrier may include a viscous material suitable for inclusion in a liquid reservoir. One example of a viscous material may include, without limitation, a material that forms a gel.

The transdermal formulations of the present invention may take numerous specific embodiments. In one aspect, the formulation may be a transdermal patch. Transdermal patches may include any type of patch known to one skilled in the art, including transdermal matrix patches, liquid reservoir patches, etc. In another aspect, the transdermal formulation may be a topical formulation. Further examples include transmucosal formulations, such as buccal and sublingual tablets or adhesive films. Topical formulations may include, without limitation, creams, lotions, ointments, gels, pastes, mousses, aerosols, sprays, waxes, balms, suppositories, and mixtures or combinations thereof. Any one of a number of specific ingredients may be used in order to provide a specifically desired transdermal formulation, such as diluents, excipients, emollients, plasticizers, skin irritation reducing agents, stabilizing compounds, and mixtures thereof.

In another aspect, a method of treating or preventing a condition in a subject for which an atomoxetine compound is effective is provided. Such a method may typically include transdermally administering a therapeutically effective amount of atomoxetine, or a transdermal atomoxetine formulation, as recited herein to the subject. Though an atomoxetine compound may prove effective in treating various conditions, particular examples may include, without limitation, attention-deficit/hyperactivity disorders (ADHD), asthma, allergic rhinitis, cognitive failure, tic disorders, depression, resistant depression with psychotic features, motor deficit after stroke, memory disorders, obesity, Tourette's syndrome, traumatic brain injury, bipolar disorder, anxiety, narcolepsy, nocturnal enuresis, fibromyalgia syndrome, schizophrenia, post traumatic stress disorder, and combinations and related disorders thereof.

In a specific embodiment of the present invention, a transdermal atomoxetine formulation is provided having a pressure sensitive acrylic polymer in an amount of about 70% w/w or greater of the transdermal formulation, atomoxetine in an amount of about 5% w/w or greater of the transdermal formulation, polyvinylpyrrolidone in an amount of about 10% w/w of the transdermal formulation, and a preferred penetration enhancer in an effective amount, and quinidine in an amount of about 0.1% w/w or greater of the transdermal formulation. Such a formulation may minimize in vivo formation of an atomoxetine metabolite in a subject.

In another specific embodiment, a transdermal atomoxetine formulation is provided having a pressure sensitive acrylic polymer in an amount of about 70% w/w or greater of the transdermal formulation, 4-hydroxyatomoxetine in an amount of about 5% w/w or greater of the transdermal formulation, polyvinylpyrrolidone in an amount of about 10% w/w of the transdermal formulation, a preferred penetration enhancer in an effective amount, and quinidine in an amount of about 0.1% w/w or greater of the transdermal formulation. Such a formulation may also minimize in vivo formation of an atomoxetine metabolite in a subject.

Various penetration enhancers are contemplated that may be utilized in aspects of the present invention. Examples may include, without limitation, one or more of the following: lower chain (C2 to C4) alcohols, lower chain diols (such as propylene glycol, di-propylene glycol), triacetin, glycerol monooleate, glycerol monolaurate, oleic alcohol, lauryl alcohol, isopropyl myrisate, sorbitan esters, other surfactant type enhancers, and additional enhancers known in the art and cited in the technical literature. Additional information regarding permeation enhancers may be found in “Skin Permeation Enhancers Cited in the Technical Literature,” Osborne, et al , Pharmaceutical Technology, June 1998, which is incorporated herein by reference in its entirety. Additionally, permeation enhancers may be utilized in an effective amount, which, for permeation enhancers, may include an amount that will increase the permeability of a drug by at least 1% to 20%.

### DETAILED DESCRIPTION

Definitions

In describing and claiming the present invention, the following terminology will be used in accordance with the definitions set forth below.

The singular forms “a,” “an,” and, “the” include plural referents unless the context clearly dictates otherwise. Thus, for example, reference to “an adhesive” includes reference to one or more of such adhesives, and reference to “an excipient” includes reference to one or more of such excipients.

As used herein, the terms “atomoxetine” and “tomoxetine” may be used interchangeably, both of which refer to a compound having the general chemical structure:

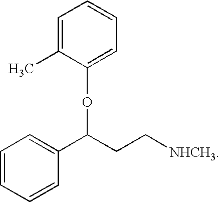

Atomoxetine is well known in the art, and is also known chemically as (−)-N-methyl-3-phenyl-3-(o-tolyloxy)-propylamine. This selective norepinephrine reuptake inhibitor is commercially available as atomoxetine HCl under the brand name Strattera® from Eli Lilly Co. Numerous metabolites of atomoxetine are known having varying physiological activities. For example, atomoxetine is converted in vivo into the active metabolite 4-hydroxyatomoxetine (4HA), primarily by aromatic hydroxylation via the cytochrome P450 2D6 (CYP2D6) enzymatic pathway. Atomoxetine is also converted in vivo into the active metabolite N-desmethylatomoxetine (NDA), primarily through the cytochrome P450 2C19 (CYP2C 19) enzymatic pathway.

As used herein in, “4-hydroxyatomoxetine” and “4HA” may be used interchangeably, and refer to a compound having the general chemical structure:

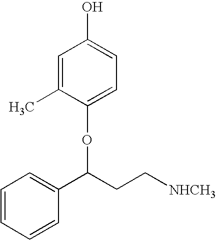

4HA possesses similar inhibitory activity to the norepinephrine reuptake transporter as atomoxetine, and is also known to be a pharmacologically active serotonin reuptake inhibitor. This metabolite appears to show little affinity to other receptor systems. 4HA is metabolized through glucuronidation to form the inactive metabolite 4-hydroxyatomoxetine-O-glucuronide (4HAO-G), which is further metabolized and/or eliminated from the body. 4HAO-G is formed to a large extent presystemically through first pass hepatic metabolism mechanisms in the gut and liver when atomoxetine compounds are administered orally.

As used herein in, “N-desmethylatomoxetine” or “NDA” may be used interchangeably, and refer to a compound having the general chemical structure:

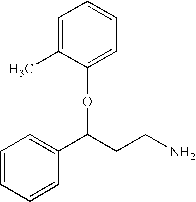

NDA is less active at inhibiting the norepinephrine reuptake transporter compared to atomoxetine. This metabolite appears to show little affinity to other receptor systems. With regard to metabolism, NDA is hydroxylated at the 4 position of the phenoxy ring, glucuronidated, and subsequently eliminated from the body.

As used herein, the “phenoxy 4 position” refers to the 4^{th} carbon of the phenoxy group of an atomoxetine compound. As an illustration, the phenoxy 4 position is marked by an X in the atomoxetine compound:

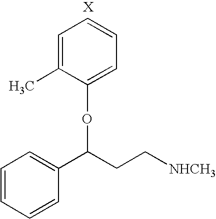

As used herein, the term “atomoxetine compound metabolite” refers to any metabolite that may be formed by metabolism of an atomoxetine compound. Atomoxetine compound metabolites may include, without limitation, 4-hydroxyatomoxetine, 4-hydroxyatomoxetine-O-glucuronide, N-desmethylatomoxetine, and combinations thereof. Various active and inactive metabolites of atomoxetine compounds are known, and it is intended that the administration of active metabolites be included in the scope of the present invention. As such, reference to minimizing the in vivo formation of an atomoxetine compound metabolite refers to the formation of a metabolite of the atomoxetine compound, whether the compound is atomoxetine, an administered metabolite, or a derivative.

As used herein, the term “atomoxetine compound” refers to atomoxetine and any functionally similar compound, including without limitation, those recited above, as well as other metabolites, derivatives, salts, prodrugs, analogs, isomers, etc.

As used herein, “subject” refers to a mammal that may benefit from the administration of a drug composition or method of this invention. Examples of subjects include humans, and may also include other animals such as horses, pigs, cattle, dogs, cats, rabbits, and aquatic mammals.

As used herein, the terms “formulation” and “composition” are used interchangeably and refer to a mixture of two or more compounds, elements, or molecules. In some aspects the terms “formulation” and “composition” may be used to refer to a mixture of one or more active agents with a carrier or other excipients. The terms “drug,” “pharmaceutical,” “active agent,” and “bioactive agent” are also used interchangeably to refer to a pharmacologically active substance or composition. These terms of art are well-known in the pharmaceutical and medicinal arts.

As used herein, “transdermal” refers to the route of administration taken by a drug that is applied to and absorbed through an area of skin. In some aspects, the skin may be substantially unbroken. Thus the terms “transdermal formulation” and “transdermal composition” can be used interchangeably, and refer to formulations or compositions that are applied to a surface of the skin and transdermally absorbed. Examples of transdermal formulations include but are not limited to, ointments, creams, gels, transdermal patches, sprays, lotions, mousses, aerosols, nasal sprays, buccal and sublingual tablets and tapes or adhesives, vaginal rings, and pastes. The term “transdermal administration” thus refers to the transdermal application of a formulation or composition. Transdermal administration can be accomplished by applying, pasting, rolling, attaching, pouring, pressing, rubbing, etc., of a transdermal preparation or formulation onto a skin or mucosal surface of a subject. These and additional methods of administration are well-known in the art.

The terms “transdermal delivery system,” “transdermal patches” or simply “patches” refer to a matrix or liquid reservoir type of transdermal delivery device which is used to transdermally deliver defined doses of a substance, over a specific application period.

By the term “matrix”, “matrix system”, or “matrix patch” is meant a composition comprising an effective amount of a drug dissolved or dispersed in a polymeric phase, often a pressure sensitive adhesive, which may also contain other ingredients, such as a penetration enhancers, skin irritation reducing agents, excipients, plasticizers, emollients, and other optional ingredients. This definition is meant to include embodiments wherein such polymeric phase is laminated to a pressure sensitive adhesive or used within an overlay adhesive.

The general structure of a matrix-type patch is known to those skilled in the art. Such structure typically includes a drug-impermeable occlusive backing laminated to the distal side of a solid or semisolid matrix layer comprised of a homogeneous blend of the drug, a polymeric pressure sensitive adhesive carrier, and optionally one or more skin penetration enhancers, and a temporary peelable release liner adhered to the proximal side of the matrix layer. In use, the release liner is removed prior to application of the patch to the skin. Matrix patches are known in the art of transdermal drug delivery. Examples without limitation, of adhesive matrix transdermal patches are those described or referred to in U.S. Pat. Nos. 5,985,317, 5,783,208, 5,626,866, 5,227,169, 5,122,383 and 5,460,820 which incorporated by reference in their entirety.

Additionally, the general structure of a liquid reservoir system (LRS) type patch is also known. Such patches typically comprise a fluid of desired viscosity, such as a gel or ointment, which is formulated for confinement in a reservoir having an impermeable backing and a skin contacting permeable membrane, or membrane adhesive laminate providing diffusional contact between the reservoir contents and the skin. The drug and any penetration enhancers are contained in the fluid in desired amounts. For application, a peelable release liner is removed and the patch is attached to the skin surface. LRS patches are known in the art of transdermal drug delivery. Examples without limitation, of LRS transdermal patches are those described or referred to in U.S. Pat. Nos. 4,849,224, 4,983,395, which are incorporated by reference in their entirety.

The terms “skin,” “skin surface,” “derma,” “epidermis,” and similar terms are used interchangeably herein, and refer to not only the outer skin of a subject comprising the epidermis, but also to mucosal surfaces to which a drug composition may be administered. Examples of mucosal surfaces include the mucosal of the respiratory (including nasal and pulmonary), oral (mouth and buccal), vaginal, introital, labial, and rectal surfaces. Hence the terms “transdermal” encompasses “transmucosal” as well.

As used herein, “enhancement,” “penetration enhancement,” or “permeation enhancement,” refer to an increase in the permeability of the skin to a drug, so as to increase the rate at which the drug permeates through the skin. Thus, “permeation enhancer,” “penetration enhancer,” or simply “enhancer” refers to an agent, or mixture of agents that achieves such permeation enhancement. Several compounds have been investigated for use as penetration enhancers. See, for example, U.S. Pat. Nos. 5,601,839; 5,006,342; 4,973,468; 4,820,720; 4,006,218; 3,551,154; and 3,472,931. An index of penetration enhancers is disclosed by David W. Osborne and Jill J. Henke, in their publication entitled Skin Penetration Enhancers Cited in the Technical Literature, published in “Pharmaceutical Technology” (June 1998), which is incorporated by reference herein.

As used herein, an “effective amount” or a “therapeutically effective amount” of a drug refers to a non-toxic, but sufficient amount of the drug, to achieve therapeutic results in treating a condition for which the drug is known to be effective. It is understood that various biological factors may affect the ability of a substance to perform its intended task. Therefore, an “effective amount” or a “therapeutically effective amount” may be dependent in some instances on such biological factors. Further, while the achievement of therapeutic effects may be measured by a physician or other qualified medical personnel using evaluations known in the art, it is recognized that individual variation and response to treatments may make the achievement of therapeutic effects a somewhat subjective decision. The determination of an effective amount is well within the ordinary skill in the art of pharmaceutical sciences and medicine. See, for example, Meiner and Tonascia, “Clinical Trials: Design, Conduct, and Analysis,” Monographs in Epidemiology and Biostatistics, Vol. 8 (1986), incorporated herein by reference. Thus, an “effective amount” of an enhancer refers to an amount sufficient to increase the penetration of a drug through the skin to a selected degree. Methods for assaying the characteristics of penetration enhancers are well-known in the art. See, for example, Merritt et al., “Diffusion Apparatus for Skin Penetration,” J. of Controlled Release 61 (1984), incorporated herein by reference in its entirety.

As used herein, “pharmaceutically acceptable carrier” and “carrier” may be used interchangeably, and refer to any inert and pharmaceutically acceptable material that has substantially no biological activity, and makes up a substantial part of the formulation. The carrier may be polymeric, such as an adhesive, or non-polymeric, and is generally admixed with other components of the composition (e.g., drug, binders, fillers, penetration enhancers, anti-irritants, emollients, lubricants, etc., as needed) to comprise the formulation.

The term “admixed” means that the drug and/or other ingredients can be dissolved, dispersed, or suspended in the carrier. In some cases, the drug may be uniformly admixed in the carrier.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result. For example, a composition that is “substantially free of” particles would either completely lack particles, or so nearly completely lack particles that the effect would be the same as if it completely lacked particles. In other words, a composition that is “substantially free of” an ingredient or element may still actually contain such item as long as there is no measurable effect thereof.

As used herein, the term “about” is used to provide flexibility to a numerical range endpoint by providing that a given value may be “a little above” or “a little below” the endpoint.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 2, 3, 4, and 5, individually.

This same principle applies to ranges reciting only one numerical value as a minimum or a maximum. Furthermore, such an interpretation should apply regardless of the breadth of the range or the characteristics being described.

The Invention

As has been described herein, metabolic conversion variabilities between individuals may affect pharmacokinetic profiles, and thus may affect therapeutic activity for both atomoxetine compounds and active atomoxetine compound metabolites. Variabilities can arise from various factors, such as CYP2D6 genetic diversity in a population or from drug-drug interactions with potent CYP2D6 inhibitors. One approach to minimizing such metabolic variabilities may include transdermal administration of atomoxetine compounds, including atomoxetine compound metabolites such as 4HA. In addition to transdermal administration that bypasses first pass hepatic metabolism, administration of a metabolite such as 4HA may bypass the CYP2D6 enzymatic pathway altogether, as CYP2D6 appears to not be a major metabolizer of 4HA. Thus, the transdermal administration of atomoxetine compounds may overcome these metabolic variabilities, and thus provide improved dosing/therapy. As such, the in vivo potency of an atomoxetine compound may be maximized by minimizing drug metabolism through transdermal administration of these compounds to a subject. Additionally, the transdermal administration of an atomoxetine compound may also reduce the drug's overall metabolic burden due to the comparatively lower administered dosage. As such, in one aspect of the present invention, the transdermal administration of an atomoxetine compound may be sufficient to provide a therapeutically effective atomoxetine blood serum level while minimizing atomoxetine metabolism.

The present invention can be used to deliver a wide variety of atomoxetine compounds to a subject. In addition to atomoxetine itself, the inventors have found that the transdermal administration of certain metabolites of atomoxetine may be particularly effective in treating ADHD and other related disorders due to their avoidance of certain primary hepatic metabolic mechanisms. Examples of specific atomoxetine compounds include, without limitation, atomoxetine, 4-hydroxy atomoxetine (4HA), N-desmethylatomoxetine (NDA), and metabolites, derivatives, salts, prodrugs, analogs, and combinations thereof. In one specific aspect, the atomoxetine compound may be atomoxetine. In another specific aspect, the atomoxetine compound may be 4HA. In yet another specific aspect, the atomoxetine compound may be NDA, particularly, due to NDA's lower inhibitory activity, for those situations where lower inhibition of the norepinephrine transporter may be desired.

In one aspect of the present invention, the atomoxetine compound may be an atomoxetine compound blocked at the phenoxy 4 position. Because atomoxetine compounds, particularly 4HA, are glucuronidated via the phenoxy 4 position and eliminated from the body, blocking this position may increase the potency of the atomoxetine compound by reducing drug metabolism and subsequent elimination. Any means of blocking the phenoxy 4 position known to one skilled in the art is considered to be within the scope of the present invention. As an illustration, the phenoxy 4 position is marked by an R in the following exemplary structure:

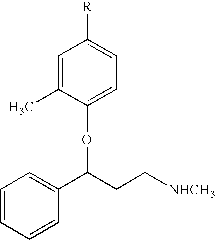

For example, the phenoxy 4 position may be blocked with an ester moiety, as illustrated in the following exemplary structure:

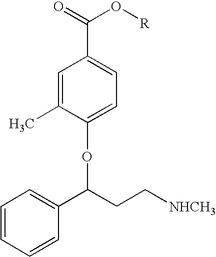

### For such moieties, R may include, without limitation, —CH₃, —C₂H₅, a substituted or unsubstituted —C₆H₉O₆ group, a branched or unbranched C₁-C₈ lower alkyl substituted or unsubstituted with halide groups, or combinations thereof.

The amount of an atomoxetine compound to be administered may be measured according to several different parameters. In one aspect, the amount of the atomoxetine compound administered may be an amount sufficient to achieve a therapeutic effect. The amount required to obtain a therapeutic effect may vary depending on a number of factors, including the activity or potency of the specific atomoxetine compound selected, as well as physiological variations among subjects as to drug tolerance and general metabolic issues. In one aspect, behavioral variation can provide some measure of therapeutic effectiveness. As such, it is well within the knowledge of those skilled in the art and in view of the present disclosure to determine dosages of atomoxetine compounds that are therapeutically effective for a given subject. In one aspect, at least about 1 mg of an atomoxetine compound can be administered to achieve therapeutic effectiveness. In another aspect, at least from about 1 mg to about 100 mgs can be administered. In yet another aspect, at least from about 2 mg to about 40 mgs can be administered. In yet another aspect, from about 2 mg to about 25 mgs can be administered.

The exact amount of an atomoxetine compound to be included in the transdermal formulations of the present invention to achieve a therapeutically effective amount can also be determined by one of ordinary skill in the art. Such a determination may depend again on the activity or potency of the specific atomoxetine compound selected and physiological variations among subjects as to drug tolerance and general metabolic issues, as well as the specific type of transdermal formulation to be employed. Further, considerations for drug load may also be made in view of specifically desired properties for the transdermal formulation, such as size, delivery rate, and duration of administration, and may range from subsaturated to supersaturated concentrations. However, in one aspect, the amount of an atomoxetine compound may be from about 0.1% w/w to about 50% w/w of the transdermal formulation. In another aspect, the atomoxetine compound may be from about 1% w/w to about 20% w/w of the transdermal formulation. In yet another aspect, the atomoxetine compound may be about 5% w/w of the transdermal formulation.

The administration dosage of the atomoxetine formulation may also be characterized in terms of blood serum levels. In one aspect, for example, atomoxetine may be transdermally administered in an amount sufficient to achieve and sustain a therapeutically effective atomoxetine blood serum level for at least about one day. In another aspect, atomoxetine may be transdermally administered in an amount sufficient to achieve and sustain a therapeutically effective atomoxetine blood serum level for less than about one day. In yet another aspect, atomoxetine may be transdermally administered in an amount sufficient to achieve and sustain a therapeutically effective atomoxetine blood serum level for from about one day to about 7 days. In a further aspect, atomoxetine may be transdermally administered in an amount sufficient to achieve and sustain a therapeutically effective atomoxetine blood serum level for from about 7 days to about 14 days. In a yet a further aspect, atomoxetine may be transdermally administered in an amount sufficient to achieve and sustain a therapeutically effective atomoxetine blood serum level for from about 1 day to about 14 days.

Any pharmaceutically acceptable transdermal formulation and method for administering an atomoxetine compound that does not interfere with the drug's therapeutic effects may be used for achieving the desired aspects of the present invention. The transdermal drug delivery system of the present invention may take a variety of well-known delivery formulations, including but not limited to, transdermal patches such as adhesive matrix patches, liquid reservoir system (LRS) patches, transmucosal patches or tablets, and topical formulations, such as creams, lotions, ointments, gels, pastes, mousses, aerosols, sprays, waxes, balms, suppositories, etc.

When presented in the form of a transdermal patch, the transdermal drug delivery system of the present invention may include various structural components, as is known in the art. For example, in the case of an adhesive matrix patch, a distal backing is often laminated to a matrix polymer layer. Such a distal backing defines the side of the matrix patch that faces the environment, i.e., distal to the skin or mucosa. The backing layer functions to protect the matrix polymer layer and drug/enhancer composition and to provide an impenetrable layer that prevents loss of drug to the environment. Thus, the material chosen for the backing should be compatible with the polymer layer, drug, and other components such as an enhancer, and should be minimally permeable to any components of the matrix patch. In one aspect, the backing may be opaque to protect components of the matrix patch from degradation from exposure to ultraviolet light. In another aspect, the backing may be transparent in order to minimize the visibility of the patch when applied. Furthermore, the backing should be capable of binding to and supporting the polymer layer, yet should be pliable enough to accommodate the movements of a person using the matrix patch.

Suitable materials for the backing include, but are not limited to: metal foils, metalized polyfoils, composite foils or films containing polyester such as polyester terephthalate, polyester or aluminized polyester, polytetrafluoroethylene, polyether block amide copolymers, polyethylene methyl methacrylate block copolymers, polyurethanes, polyvinylidene chloride, nylon, silicone elastomers, rubber-based polyisobutylene, styrene, styrene-butadiene and styrene-isoprene copolymers, polyethylene, and polypropylene. Additionally, the backing may include various foams, such as closed cell foams. Examples may include, without limitation, polyolefin foams, polyvinyl chloride foams, polyurethane foams, polyethylene foams, etc. In one aspect of the invention, the backing layer may have a thickness of about 0.0005 to 0.1 inch.

In one general aspect, the transdermal drug delivery system of the present invention can comprise a pharmaceutically acceptable carrier intended to contain the atomoxetine compound and any other components included in the formulation. A number of pharmaceutically acceptable carriers are known to those of ordinary skill in the art and may be used in connection with the present invention.

Further, a release liner may be temporarily provided upon the proximal side (side to adhere to the skin) of an adhesive layer. Such a liner provides many of the same functions as the backing layer, prior to adhesion of the patch to the skin. In use, the release liner is peeled from the adhesive layer just prior to application and discarded. The release liner can be made of the same materials as the backing layer, or other suitable films coated with an appropriate release surface.

Pharmaceutically acceptable carriers for use when the transdermal formulations of the present invention take the embodiment of an LRS patch may be any suitable viscous material known to those skilled in the art of transdermal drug delivery. Such carriers are typically a fluid of desired viscosity, such as a gel or ointment, which is formulated for confinement in a reservoir having an impermeable backing and a skin contacting permeable membrane, or membrane adhesive laminate providing diffusional contact between the reservoir contents and the skin. Such a viscous carrier may contain the atomoxetine compound to be transdermally delivered, as well as other optional components of the transdermal formulation.

Pharmaceutically acceptable carriers suitable for use when the present invention takes the embodiment of a transdermal matrix patch are also known to those of ordinary skill in the art. The present invention contemplates various structural types of transdermal matrix patches. For example, monolithic systems where the drug and enhancer are contained directly in a single pressure sensitive adhesive layer, as well as systems containing one or more polymeric reservoirs in addition to a pressure sensitive adhesive layer may be utilized. In aspects comprising systems having multiple layers/laminates, a rate controlling member may be included. Generally, a rate controlling member is located between a reservoir layer and the skin. In those aspects including a delivery layer and a reservoir layer, the rate controlling member may be adhered between a proximal side of the reservoir layer, and a distal side of the delivery layer. The rate controlling member is provided for the purpose of metering, or controlling, the rate at which drug and/or penetration enhancer migrates from the storage layer into the delivery layer. As noted herein, in one aspect of the present invention, various levels of permeation enhancement may be used to increase the delivery rate of the drug, and thus be used to vary other parameters, such as patch size, etc.

In one aspect, the pharmaceutically acceptable carrier used in a matrix patch can be a biocompatible polymer. Various general categories of biocompatible polymers are known, including, without limitation, rubbers; silicone polymers and copolymers; acrylic polymers and copolymers; and mixtures thereof. In one aspect, the biocompatible polymer can be a rubber, including natural and synthetic rubbers. One specific example of a useful rubber is a plasticized styrene-rubber block copolymer. In another aspect, the biocompatible polymer can include silicon polymers, polysiloxanes, and mixtures thereof. In yet another aspect, the biocompatible polymer can include acrylic polymers, polyacrylates, and mixtures thereof. In a further aspect, the biocompatible polymer can include vinyl acetates, ethylene-vinyl acetate copolymers, polyurethanes, plasticized polyether block amide copolymers, and mixtures thereof. In one specific aspect, the biocompatible polymer can include an acrylic copolymer adhesive such as copolymers of 2-ethylhexyacrylate and n-vinyl pyrrolidone adhesives.

In one aspect, the biocompatible polymer of the pharmaceutically acceptable carrier can be suitable for long-term (e.g., greater than 1 day, maybe about 3-4 days, or longer such as 7 days, or even 1-4 weeks) contact with the skin. In another aspect, the biocompatible polymer of the carrier is suitable for a short-term administration (e.g., for a few minutes to a few hours, less than or equal to 1 day). Such biocompatible polymers must be physically and chemically compatible with the atomoxetine compound, and with any carriers and/or vehicles or other additives incorporated into the formulation. In one aspect of the invention, the biocompatible polymers of the pharmaceutically acceptable carrier can include polymeric adhesives. Example of such adhesives can include without limitation, acrylic adhesives including cross-linked and uncross-linked acrylic copolymers; vinyl acetate adhesives; natural and synthetic rubbers including polyisobutylenes, neoprenes, polybutadienes, and polyisoprenes; ethylenevinylacetate copolymers; polysiloxanes; polyacrylates; polyurethanes; plasticized weight polyether block amide copolymers, and plasticized styrene-rubber block copolymers or mixtures thereof. In a further aspect of the invention, contact adhesives for use in the pharmaceutically acceptable carrier layer are acrylic adhesives, such as DuroTak™ 87-2888 adhesive (National Starch & Chemical Co., Bridgewater, N.J.); and polyisobutylene adhesives such as ARcare™. MA-24 (Adhesives Research, Glen Rock, Pa.) and ethylene vinyl acetate copolymer adhesives. In yet another aspect, gel-type or “hydrogel” adhesives are contemplated for use. See for example, U.S. Pat. No. 5,827,529 which is incorporated herein by reference. Those of ordinary skill in the art will appreciate that the specific type and amount of adhesive polymer used may be selected depending upon the desired specific characteristics of the final product. However, in one aspect, the amount of adhesive polymer in the adhesive matrix layer may be at least about 50% w/w of the adhesive layer. In another aspect, the amount may be at least about 60% w/w of the adhesive layer. In yet another aspect, the amount may be at least about 85% w/w of the adhesive layer. In a further aspect, the amount may be at least about 90% w/w of the adhesive layer. In an additional aspect, the amount may be from about 50% w/w to about 95% w/w of the adhesive layer.

Transdermal matrix patches may be utilized in various sizes, depending on the atomoxetine dosage in the patch and the desired rate of delivery. In one aspect, transdermal patches may be from about 0.5 cm² to about 200 cm² in size. In another aspect, transdermal patches may be from about 5 cm² to about 75 cm² in size. In yet another aspect, transdermal patches may be from about 10 cm² to about 100 cm² in size. In a further aspect, transdermal patches may be from about 50 cm² to about 100 cm² in size. In yet a further aspect, transdermal patches may be from about 0.5 cm² to about 100 cm² in size. In an additional aspect, transdermal patches may be from about 100 cm² to about 200 cm² in size. In yet an additional aspect, transdermal patches may be from about 10 cm² to about 50 cm² in size.

Various pharmaceutically acceptable carriers which are known to those of ordinary skill in the art may be used when the transdermal formulations of the present invention take the embodiment of a topical formulation. In one aspect, the topical carrier can be an ointment including an atomoxetine compound. An ointment is a semisolid pharmaceutical preparation based on well known materials such as oleaginous bases, lanolins, emulsions, or water-soluble bases. Preparation of ointments is well known in the art such as described in Remington: The Science and Practice of Pharmacy 19^{th} ed. (1995), vol. 2, pp. 1585-1591, which is incorporated herein by reference. Such preparations often contain petrolatum or zinc oxide together with a drug. Oleaginous ointment bases suitable for use in the present invention include generally, but are not limited to, vegetable oils, animal fats, and semisolid hydrocarbons obtained from petroleum. Absorbent ointment bases of the present invention may contain little or no water and may include components such as, but not limited to, hydroxystearin sulfate, anhydrous lanolin and hydrophilic petrolatum. Emulsion ointment bases of the present invention are either water-in-oil (W/O) emulsions or oil-in-water (O/W) emulsions, and may include, but are not limited to, cetyl alcohol, glyceryl monostearate, lanolin, polyalkylsiloxanes, and stearic acid. Water-soluble ointment bases suitable for use in the present invention may be prepared from polyethylene glycols of varying molecular weight.

In another aspect of the present invention, the topical carrier can be a cream including an atomoxetine compound. Creams are a type of ointment which are viscous liquids or semisolid emulsions, either oil-in-water or water-in-oil, as is well known in the art. Cream bases may be soluble in water, and contain an oil phase, an emulsifier, an aqueous phase, and the active agent. In a detailed aspect of the present invention, the oil phase may be comprised of petrolatum and a fatty alcohol such as cetyl or stearyl alcohol. In another detailed aspect of the present invention, the aqueous phase may exceed the oil phase in volume, and may contain a humectant. In another detailed aspect of the present invention, the emulsifier in a cream formulation may be a nonionic, anionic, cationic or amphoteric surfactant.

In another aspect of the present invention, the topical carrier can be a lotion including an atomoxetine compound. A lotion is an ointment which may be a liquid or semi-liquid preparation in which solid particles, including the active agent, are present in a water or alcohol base. Lotions suitable for use in the present invention may be a suspension of solids or may be an oil-in-water emulsion. In another aspect of the present invention, lotions may also contain suspending agents which improve dispersions or other compounds which improve contact of the active agent with the skin, e.g., methylcellulose, sodium carboxymethylcellulose, or similar compounds.

In yet another aspect of the present invention, a topical carrier can be a paste including an atomoxetine compound. Pastes of the present invention are ointments in which there are significant amounts of solids which form a semisolid formulation in which the active agent is suspended in a suitable base. In a detailed aspect of the present invention, pastes may be formed of bases to produce fatty pastes or made from a single-phase aqueous gel. Fatty pastes suitable for use in the present invention may be formed of a base such as petrolatum, hydrophilic petrolatum or the like. Pastes made from single-phase aqueous gels suitable for use in the present invention may incorporate cellulose based polymers such as carboxymethylcellulose or the like as a base.

In another aspect of the present invention, a topical gel may be prepared that includes an atomoxetine compound. A gel prepared in accordance with the present invention may be a preparation of a colloid in which a disperse phase has combined with a continuous phase to produce a viscous product. The gelling agent may form submicroscopic crystalline particle groups that retain the solvent in the interstices. As will be appreciated by those working in art, gels are semisolid, suspension-type systems. Single-phase gels can contain organic macromolecules distributed substantially uniformly throughout a carrier liquid, which may be aqueous or non-aqueous and may contain an alcohol or oil.

In addition to containing an atomoxetine compound, the pharmaceutically acceptable carriers of the transdermal formulations recited herein, may include a number of other additives, such as diluents, penetration enhancers, excipients, emollients, plasticizers, skin irritation reducing agents, stabilizing compounds, or a mixture thereof. These types of components, as well as others not specifically recited, are well known in the art for inclusion in various transdermal formulations, and may be added as desired to the transdermal drug delivery system of the present invention in specific types and amounts in order to achieve a desired result.

Furthermore, when the atomoxetine compound to be delivered is susceptible to acid catalyzed degradation, carriers that contain no acid functional groups, and that do not form any acid functional groups upon storage can be used in order to improve the stability of the formulation. One specific example of such a carrier is an ethylhexylacrylate polymer, as described in U.S. Pat. No. 5,780,050, which is incorporated by reference herein.

In addition to the atomoxetine compound, the transdermal formulations of the present invention may also include a penetration enhancer, or mixture of penetration enhancers in order to increase the permeability of the skin to the atomoxetine compound. For example, useful penetration enhancers may include, without limitation, fatty acids, fatty acid esters, fatty alcohols, fatty acid esters of lactic acid or glycolic acid, glycerol tri-, di-, and monoesters, triacetin, short chain alcohols, and mixtures thereof. In one specific aspect, the penetration enhancer may include lauryl alcohol, isopropyl myristate, or a combination of lauryl alcohol and isopropyl myristate. In other aspects, specific species or combinations of species may be selected from the above listed classes of compounds by one skilled in the art, in order to optimize enhancement of the particular atomoxetine compound employed.

The formulations of the present invention may also include metabolic inhibitors to increase the potency of the administered atomoxetine compound. Because atomoxetine compounds appear to be primarily metabolized by various cytochrome P450 enzymes, selective inhibition of certain enzymes may thus increase the potency of the administered atomoxetine compound by reducing metabolic activity. As such, in one aspect, a P450-mediated reaction inhibitor may be administered to a subject. The P450-mediated reaction can be any enzymatic pathway responsible for metabolism on an atomoxetine compound. Furthermore, the particular P450-mediated reaction may vary depending on the particular atomoxetine compound administered. Thus the inhibitor can be any inhibitor known to reduce the activity of the particular P450-mediated reaction. For example, and without limitation, CYP2A6 may be inhibited by coumarin, CYP2C9 by sulfaphenazole, CYP2C19 by S-mephenytoin, CYP2D6 by quinidine, CYP3A by ketoconazole, etc. In one aspect, quinidine may be useful as a P450-mediated reaction inhibitor due to the enzymatic activity of CYP2D6 in metabolizing various atomoxetine compounds.

Various temporal orders of administering the atomoxetine compound and the inhibitor are possible, and any such order of administration that obtains a therapeutically result is considered to be within the scope of the present invention. In one aspect, the atomoxetine compound and the inhibitor can be administered concomitantly, either as a single composition or as separate compounds. Such concurrent administration is intended to include application of each of the compounds at essentially the same time. In such concurrent administration, the inhibitor can be delivered concomitantly with, or separately from the atomoxetine compound. For the case of concomitant administration of the inhibitor and the drug, the inhibitor can be admixed with the drug or administered as separate compounds. In the case of separate administration of the inhibitor with respect to the drug, the inhibitor can be administered prior to, following, or both prior to and following the administration of the drug.

The transdermal formulations of the present invention can be formulated to as sustained release formulations that administer therapeutically effective amounts of a drug over an extended period of time. As such, in one aspect, the sustained delivery period of the atomoxetine may be for at least 7 days. In another aspect, the sustained delivery period may be at least 5 days. In a further aspect, the sustained delivery period may be at least 3 days. In another aspect, the sustained delivery period may be at least one day. In yet another aspect, the sustained delivery period may be less than one day. In a further aspect, the sustained delivery period may be from about 1 to about 4 weeks.

### EXAMPLES

The following examples of transdermal formulations of atomoxetine are provided to promote a more clear understanding of certain embodiments of the present invention, and are in no way meant as a limitation thereon.

### Example 1

### Preparation of Atomoextine Adhesive Matrix Patch

A general method of preparing transdermal adhesive matrix patches is described by U.S. Pat. Nos. 5,227,169, and 5,212,199, which are incorporated by reference in their entirety. Following this general method, the atomoxetine patches of this invention are prepared as follows:

Atomoxetine, triacetin (Eastman Chemical Co., Kingsport, N.Y.) and 87-2888 acrylic copolymer adhesives (National Starch and Chemical Co., Bridgewater, N.J.) are mixed into a homogenous solution and coated at 6 mg/cm² (dried weight) onto a silicone treated polyester release liner (Rexham Release, Chicago, Ill.) using a two zone coating/drying/laminating oven (Kraemer Koating, Lakewood, N.J.) to provide a final atomoxetine adhesive matrix containing 15.4%, 9.0%, and 75.6% by weight atomoxetine, triacetin and acrylic copolymer adhesive, respectively. A fifty micron thick polyethylene backing film (3M, St. Paul, Minn.) is subsequently laminated onto the dried adhesive surface of the atomoxetine containing adhesive matrix and the final laminate structure is die cut to provide patches ranging in size from 13 cm² to 39 cm² patches.

### Example 2

### Preparation of Topical Atomoxetine Formulation

Topically applied atomoxetine containing gel may be used to deliver atomoxetine in accordance with the method of the present invention. A general method of preparing a topical gel is known in the art. Following this general method, a topical gel comprising atomoxetine is prepared as follows:

95% ethanol (USP) is diluted with water (USP), glycerin (USP), and glycerol monooleate (Eastman Chemical, Kingsport N.Y.) to provide a final solution at ethanol/water/glycerin/glycerol monooleate percent ratios of 35/59/5/1, respectively. Atomoxetine is then dissolved into the above solution to a concentration of 10 mg/gram. The resultant solution is then gelled with 1% hydroxypropyl cellulose (Aqualon, Wilmington, Del.) to provide a final atomoxetine gel. One to two grams of the above gel is applied topically to approximately 200 cm² surface area on the chest, torso, and or arms to provide topical administration of atomoxetine.

It is to be understood that the above-described compositions and modes of application are only illustrative of preferred embodiments of the present invention. Numerous modifications and alternative arrangements may be devised by those skilled in the art without departing from the spirit and scope of the present invention and the appended claims are intended to cover such modifications and arrangements. Thus, while the present invention has been described above with particularity and detail in connection with what is presently deemed to be the most practical and preferred embodiments of the invention, it will be apparent to those of ordinary skill in the art that numerous modifications, including, but not limited to, variations in size, materials, shape, form, function and manner of operation, assembly and use may be made without departing from the principles and concepts set forth herein.

What is claimed is:

1. A method of maximizing in-vivo potency of an atomoxetine compound in a subject comprising:
transdermally administering the atomoxetine compound to the subject.

2. The method of claim 1, wherein the in-vivo potency of the atomoxetine compound is maximized by minimizing in vivo conversion of the atomoxetine compound to an atomoxetine compound metabolite.

3. The method of claim 2, wherein the atomoxetine compound metabolite is selected from the group consisting of 4-hydroxyatomoxetine, 4-hydroxyatomoxetine-O-glucuronide, N-desmethylatomoxetine, and combinations thereof.

4. The method of claim 1, wherein the atomoxetine compound is selected from the group consisting of atomoxetine, 4-hydroxyatomoxetine, N-desmethylatomoxetine, and metabolites, derivatives, salts, prodrugs, analogs, and combinations thereof.

5. The method of claim 4, wherein the atomoxetine compound is atomoxetine.

6. The method of claim 4, wherein the atomoxetine compound is 4-hydroxyatomoxetine.

7. The method of claim 4, wherein the atomoxetine compound is N-desmethylatomoxetine.

8. The method of claim 1, further comprising administering a P450-mediated reaction inhibitor.

9. The method of claim 8, wherein the P450-mediated reaction inhibitor is a CYP2D6 inhibitor.

10. The method of claim 8, wherein the P450-mediated reaction inhibitor is administered either prior to, concurrently with, or following the atomoxetine compound.

11. The method of claim 10, wherein the P450-mediated reaction inhibitor is administered concurrently with the atomoxetine compound.

12. The method of claim 11, wherein the P450-mediated reaction inhibitor and the atomoxetine compound are administered as a single composition.

13. The method of claim 8, wherein the P450-mediated reaction inhibitor is administered both prior to and following the atomoxetine compound.

14. A transdermal atomoxetine formulation for use in accordance with the method of claim 1 comprising:
a therapeutically effective amount of an atomoxetine compound in combination with a pharmaceutically acceptable transdermal carrier, wherein administration of the combination to a subject maximizes in vivo potency of the atomoxetine compound by minimizing metabolism thereof.

15. The transdermal atomoxetine formulation of claim 14, wherein the atomoxetine compound is selected from the group consisting of atomoxetine, 4-hydroxyatomoxetine, N-desmethylatomoxetine, and metabolites, derivatives, salts, prodrugs, analogs, and combinations thereof.

16. The transdermal atomoxetine formulation of claim 15, wherein the atomoxetine compound is atomoxetine.

17. The transdermal atomoxetine formulation of claim 15, wherein the atomoxetine compound is 4-hydroxyatomoxetine.

18. The transdermal atomoxetine formulation of claim 15, wherein the atomoxetine compound is N-desmethylatomoxetine.

19. The transdermal atomoxetine formulation of claim 1, wherein the atomoxetine compound is blocked at a phenoxy 4 position.

20. The transdermal atomoxetine formulation of claim 19, wherein the atomoxetine compound is blocked at the phenoxy 4 position with an ester moiety.

21. The transdermal atomoxetine formulation of claim 20, wherein the ester moiety is selected from the group consisting of methoxy, ethoxy, a substituted ester group of a branched or unbranched C₁-C₁₈ lower alkyl substituted or unsubstituted with halide groups, a substituted or unsubstituted phenyl, and combinations thereof.

22. The transdermal atomoxetine formulation of claim 1, wherein the pharmaceutically acceptable carrier is a biocompatible polymer.

23. The transdermal atomoxetine formulation of claim 22, wherein the biocompatible polymer is a member selected from the group consisting of: rubbers; silicone polymers and copolymers; acrylic polymers and copolymers; and mixtures thereof.

24. The transdermal atomoxetine formulation of claim 23, wherein the biocompatible polymer is a rubber selected from the group consisting of: natural and synthetic rubbers, plasticized styrene-rubber block copolymers, and mixtures thereof.

25. The transdermal atomoxetine formulation of claim 22, wherein the biocompatible polymer is a member selected from the group consisting of: silicone polymers, polysiloxanes, and mixtures thereof.

26. The transdermal atomoxetine formulation of claim 22, wherein the biocompatible polymer is a member selected from the group consisting of: acrylic polymers, polyacrylates, and mixtures thereof.

27. The transdermal atomoxetine formulation of claim 22, wherein the biocompatible polymer is a member selected from the group consisting of: vinyl acetates, ethylene-vinyl acetate copolymers, polyurethanes, plasticized polyether block amide copolymers, and mixtures thereof.

28. The transdermal atomoxetine formulation of claim 14, wherein the pharmaceutically acceptable carrier comprises a viscous material suitable for use as a liquid reservoir.

29. The transdermal atomoxetine formulation of claim 28, wherein the viscous material forms a gel.

30. The transdermal atomoxetine formulation of claim 14, further comprising an ingredient selected from the group consisting of: diluents, excipients, emollients, plasticizers, skin irritation reducing agents, stabilizing compounds, and mixtures thereof.

31. The transdermal atomoxetine formulation of claim 14, wherein the formulation is a transdermal patch.

32. The transdermal atomoxetine formulation of claim 31, wherein the transdermal patch is a transdermal matrix patch.

33. The transdermal atomoxetine formulation of claim 31, wherein the transdermal patch is a liquid reservoir patch.

34. The transdermal atomoxetine formulation of claim 14, wherein the formulation is a topical formulation.

35. The transdermal atomoxetine formulation of claim 34, wherein the topical formulation is in a form selected from the group consisting of creams, lotions, ointments, gels, pastes, mousses, aerosols, sprays, waxes, balms, suppositories, and mixtures or combinations thereof.

36. The transdermal atomoxetine formulation of claim 14, wherein the atomoxetine compound may be from about 0.1% w/w to about 50% w/w of the transdermal formulation.

37. The transdermal atomoxetine formulation of claim 36, wherein the atomoxetine compound is from about 1% w/w to about 20% w/w of the transdermal formulation.

38. The transdermal atomoxetine formulation of claim 37, wherein the atomoxetine compound is about 5% w/w of the transdermal formulation.

39. The transdermal atomoxetine formulation of claim 14, further comprising a P450-mediated reaction inhibitor.

40. The transdermal atomoxetine formulation of claim 39, wherein the P450-mediated reaction inhibitor is a CYP2D6 inhibitor.

41. The transdermal atomoxetine formulation of claim 39, wherein the P450-mediated reaction inhibitor and the atomoxetine compound are a single composition.

42. A method of treating or preventing a condition in a subject for which an atomoxetine compound is effective, comprising:
transdermally administering a therapeutically effective amount of a transdermal atomoxetine formulation as recited in claim 14 to the subject.

43. The method of claim 42, wherein the condition is selected from the group consisting of attention deficit/hyperactivity disorder, asthma, allergic rhinitis, cognitive failure, tic disorders, depression, resistant depression with psychotic features, motor deficit after stroke, memory disorders, obesity, Tourette's syndrome, traumatic brain injury, bipolar disorder, anxiety, narcolepsy, nocturnal enuresis, fibromyalgia syndrome schizophrenia, post traumatic stress disorder, and combinations and related disorders thereof.

44. The method of claim 43, wherein the condition is attention deficit/hyperactivity disorder.

45. A transdermal atomoxetine formulation, comprising:
a pressure sensitive acrylic polymer in an amount of about 70% w/w of the transdermal formulation;
atomoxetine in an amount of about 5% w/w of the transdermal formulation;
polyvinylpyrrolidone in an amount of about 10% w/w of the transdermal formulation;
a penetration enhancer in an amount of about 20% w/w of the transdermal formulation selected from the group consisting of lower chain (C2 to C4) alcohols, lower chain diols such as propylene glycol and di-propylene glycol, triacetin, glycerol monooleate, glycerol monolaurate, oleic alcohol, lauryl alcohol, isopropyl myrisate, sorbitan esters, and combinations thereof; and
quinidine in an amount of about 0.1% w/w or greater of the transdermal formulation, the formulation minimizing in vivo formation of an atomoxetine metabolite in a subject.

46. A transdermal atomoxetine formulation, comprising:
a pressure sensitive acrylic polymer in an amount of about 70% w/w of the transdermal formulation;
4-hydroxyatomoxetine in an amount of about 5% w/w of the transdermal formulation;
polyvinylpyrrolidone in an amount of about 10% w/w of the transdermal formulation;
a penetration enhancer in an amount of about 20% w/w of the transdermal formulation selected from the group consisting of lower chain (C2 to C4) alcohols, lower chain diols such as propylene glycol and di-propylene glycol, triacetin, glycerol monooleate, glycerol monolaurate, oleic alcohol, lauryl alcohol, isopropyl myrisate, sorbitan esters, and combinations thereof; and
quinidine in an amount of about 0.1% w/w or greater of the transdermal formulation, the formulation minimizing in vivo formation of an atomoxetine metabolite in a subject.
